(19) United States
(12) Patent Application Publication
(10) Pub. No.: US 2020/0011860 A1
(43) Pub. Date: Jan. 9, 2020

(54) Functionalized Sensor for Detection of Biomarkers

(71) Applicants: Namal Nawana, Weston, MA (US); Rite Taste, LLC, Weston, MA (US)

(72) Inventors: Namal Nawana, Weston, MA (US); Mohammad E. Taslim, Needham, MA (US); Mohammed Fotouhi, Weston, MA (US); Mehdi Abedi, Brighton, MA (US); Reza Mollaaghababa, Natick, MA (US); Edward Alvin Greenfield, Stoughton, MA (US)

(21) Appl. No.: 16/422,743

(22) Filed: May 24, 2019

Related U.S. Application Data
(60) Provisional application No. 62/676,079, filed on May 24, 2018.

(51) Int. Cl. G01N 33/543; B01L 3/00; G01N 27/12

(52) U.S. Cl. CPC G01N 33/5438; B01L 3/508; B01L 2200/143; B01L 2300/12; B01L 2300/0645; G01N 27/125

(57) ABSTRACT

In one aspect, the present teachings provide a sensor for detecting troponin, e.g., cardiac troponin in a patient's blood, that relies on an electronic signature generated via interaction of troponin with an anti-troponin antibody. More specifically, as discussed in more detail below, such a sensor can include a graphene layer disposed on an underlying substrate, where the graphene layer has been functionalized with an anti-troponin antibody. The interaction of troponin in a sample, e.g., a patient's blood, with the anti-troponin antibody coupled, e.g., anchored, to the graphene substrate, can modulate an electronic property of the underlying graphene layer. The modulation of the electronic property of the graphene layer can be measured and used to identify, and in some embodiments quantify, troponin in the sample.

### RELATED APPLICATION

The present application claims priority to Provisional Application No. 62/676,079 titled “A Graphene-Functionalized Sensor,” which was filed on May 24, 2018 and which is herein incorporated by reference in its entirety.

### BACKGROUND

The present invention relates generally to sensing systems and methods for detecting, and optionally quantifying, troponin protein in a sample, e.g., blood plasma.

Troponin is a complex of three regulatory proteins, namely, troponin C (the calcium binding element), troponin I (the actinomyosin binding element), and troponin T (the tropomyosin binding element), which play an important role in muscle contraction in skeletal muscle and cardiac muscle. The ICT troponin complex serves to regulate the calcium-dependent interaction of myosin and actin. Troponin I and T exist in three distinct molecular forms, which correspond to specific isoforms found in fast-twitch skeletal muscle, slow-twitch skeletal muscle, and heart tissue.

Troponin is known as a useful marker for various heart disorders, and in particular, as a highly specific marker for myocardial infarction. In particular, certain types of troponin (cardiac I and T) can be used as highly specific indicators of damage to the heart muscle (myocardium). For example, the level of these types of troponin in a patient's blood can be used to differentiate between unstable angina and myocardial infarction in people with chest pain or acute coronary syndrome. After myocardial infarction, an elevated level of troponin can be detected in a patient's blood after 2-4 hours. The elevated troponin level can persist for up to 5-14 days. A level of troponin of 2 micrograms or more is currently utilized as the criterion for indicating myocardial infarction.

Conventional methods for detecting and quantifying cardiac troponin in blood include immunoassays, such as ELISA, that utilize a variety of detection techniques, such as fluorometric detection, chemiluminescence detection, electrochemical detection, etc. The conventional methods can be time-consuming and/or can require complex sample preparation.

Accordingly, there is a need for improved systems and methods for detecting troponin in a sample.

### SUMMARY

In one aspect, a sensor for detecting a biomarker in a sample is disclosed, which comprises a graphene layer, a plurality of antibodies coupled to said graphene layer, where the antibodies exhibit specific binding to the biomarker. A reference electrode is disposed in vicinity of the graphene layer. An AC (alternating current) voltage source can be employed for applying an AC voltage to the reference electrode so as to generate a time-varying electric field at or near vicinity of the graphene surface. A plurality of electrical conductors are electrically coupled to the antibody-functionalized graphene layer for measuring an electrical property of the functionalized graphene layer.

In some embodiments, the AC voltage source is configured to apply an AC voltage with a frequency in a range of about 1 kHz to about 1 MHz to the reference electrode. In some embodiments, the applied AC voltage has an amplitude in a range of about 1 millivolt to about 3 volts.

In some embodiments, the biomarker can be any of troponin, C-reactive protein, B-type natriuretic peptide, myeloperoxidase, and creatine kinase MB.

In a related aspect, a system for detecting a panel of biomarkers in a biological sample is disclosed. Which comprises a plurality of sensors each configured to detect one of a plurality of biomarkers. Each of the sensors comprises a graphene layer disposed on an underlying substrate, a plurality of antibodies coupled to said graphene layer, wherein said antibodies exhibit specific binding to one of said plurality of biomarkers. A plurality of electrical conductors are electrically coupled to the antibody-functionalized graphene layer to measure at least one electrical property of the antibody-functionalized graphene layer.

In some embodiments of the above system, each of the sensors is configured to detect a different one of the panel of the biomarkers. In some embodiments, at least one of the sensors is configured for detecting troponin in the biological sample. In some embodiments, at least one of the sensors is configured for detecting C-reactive protein in the biological sample. In some embodiments, at least one of the sensors is configured for detecting B-type natriuretic peptide in said biological sample. In some embodiments, at least one of the sensors is configured for detecting myeloperoxidase in the biological sample. In some embodiments, at least one of the sensors is configured for detecting creatine kinase MB in the biological sample.

In a related aspect, a sensor for detecting a biomarker in a biological sample is disclosed, which comprises a test sensing unit configured for receiving the biological sample and detecting the biomarker in the biological sample if a concentration of the biomarker in the sample is above a threshold (e.g., about 2 micrograms), a calibration sensing unit configured for receiving a calibration sample for providing calibration data for use in quantifying the biomarker detected by said test sensing unit. Each of the test sensing unit and the calibration sensing unit comprises a graphene layer disposed on an underlying substrate, and a plurality of antibodies coupled to said graphene layer, wherein said antibodies exhibit specific binding to said biomarker, and a plurality of electrical conductors electrically coupled to said functionalized graphene layer for measuring an electrical property of said functionalized graphene layer.

In some embodiments, the biomarker includes any of troponin, C-reactive protein, B-type natriuretic peptide, and myeloperoxidase. Further, the biological sample can include any of blood, urine, saliva, spinal fluid, vaginal secretions, fecal slurries, and pleural lavage fluids, among others.

In some embodiments, an analyzer can be coupled to the sensor for receiving data from said test sensing unit and said calibration sensing unit and operating on said data to quantify amount of the biomarker in the biological sample.

In some embodiments, each of the test sensing unit and the calibration testing unit includes a reference electrode that is positioned in proximity of the graphene layer. An AC voltage source can be provided for applying an AC voltage to the reference electrode. In some embodiments, the AC voltage can have a frequency in a range of about 1 kHz, to about 1 MHz, and an amplitude in a range of about 1 millivolt to about 3 volts.

In one aspect, the present teachings provide a sensor for detecting troponin, e.g., cardiac troponin in a patient's blood, that relies on an electrical signature generated via interaction of troponin with an anti-troponin antibody. More specifically, as discussed in more detail below, such a sensor can include a graphene layer disposed on an underlying substrate, where the graphene layer has been functionalized with an anti-troponin antibody. The interaction of troponin in a sample, e.g., a patient's blood, with the anti-troponin antibody coupled, e.g., anchored, to the graphene substrate, can modulate an electrical property of the underlying graphene layer. The modulation of the electrical property of the graphene layer can be measured and used to identify, and in some embodiments quantify, troponin in the sample.

In one aspect, a sensor for detecting troponin in a sample, e.g., blood plasma, is disclosed, which comprises a functionalized graphene layer, which includes a graphene layer and a plurality of anti-troponin antibodies coupled to the graphene layer. In some embodiments, a linker is employed to couple the anti-troponin antibody to the graphene layer. For example, the linker can be covalently bound to the graphene layer and the anti-troponin antibody can be covalently bound to the linker. A plurality of electrical conductors are coupled to the functionalized graphene layer for measuring an electrical property of that layer in response to interaction of the layer with a sample. By way of example, in some embodiments, the electrical property is the DC (direct current) electrical resistance of the functionalized graphene layer.

In some embodiments, the anti-troponin antibodies comprise antibodies that exhibit specific binding to any of cardiac troponin I (cTNI) or cardiac troponin T (cTNT).

In some embodiments, the graphene layer can be disposed on an underlying substrate, such as a silicon or a polymeric substrate.

In another aspect, a sensor for detecting troponin in a sample is disclosed, which comprises at least two sensing elements, each configured for detecting a different type of troponin. One sensing element can include a graphene layer functionalized with an antibody configured to specifically bind to one type of troponin and another sensing element can include a graphene layer functionalized with an antibody configured to specifically bind to a different type of troponin.

In a related aspect, a method of detecting troponin in a sample is disclosed, which comprises applying the sample to a graphene layer functionalized with a plurality of anti-troponin antibodies, measuring at least one electrical property of the functionalized graphene layer, and using said measured electrical property to determine whether troponin is present in said sample. The method can further comprise quantifying troponin present in the sample. By way of example, the sample can be a patient's blood. In some embodiments, the measured electrical property of the functionalized graphene layer can include a DC electrical resistance thereof. In some embodiments, the step of using the measured electrical property comprises monitoring a change in said electrical property in response to interaction of the sample with the functionalized graphene layer.

In one aspect, a sensor is disclosed, which comprises a graphene layer and a plurality of anti-CRP (C reactive protein) antibodies coupled to the graphene layer. The sensor further includes a plurality of electrical conductors electrically coupled to said functionalized graphene layer for measuring an electrical property of said functionalized graphene layer in response to the interaction of a sample under study with the functionalized graphene layer.

In another aspect, a sensor is disclosed, which comprises a graphene layer and a plurality of anti-BNP (B-type natriuretic peptide) antibodies coupled to the graphene layer. The sensor further includes a plurality of electrical conductors electrically coupled to said functionalized graphene layer for measuring an electrical property of said functionalized graphene layer in response to the interaction of a sample under study with the functionalized graphene layer.

In another aspect, a sensor is disclosed, which comprises a graphene layer and a plurality of anti-CK-MB (creatine kinase MB) antibodies coupled to the graphene layer. The sensor further includes a plurality of electrical conductors electrically coupled to said functionalized graphene layer for measuring an electrical property of said functionalized graphene layer in response to the interaction of a sample under study with the functionalized graphene layer.

In another aspect, a sensor is disclosed, which comprises a graphene layer and a plurality of anti-myoglobin antibodies coupled to the graphene layer. The sensor further includes a plurality of electrical conductors electrically coupled to said functionalized graphene layer for measuring an electrical property of said functionalized graphene layer in response to the interaction of a sample under study with the functionalized graphene layer.

In another aspect, a sensor is disclosed, which comprises a graphene layer and a plurality of anti-MP (myeloproxidase) antibodies coupled to the graphene layer. The sensor further includes a plurality of electrical conductors electrically coupled to said functionalized graphene layer for measuring an electrical property of said functionalized graphene layer in response to the interaction of a sample under study with the functionalized graphene layer.

In a related aspect, a sensor is disclosed, which comprises a plurality of sensing elements, where each of the sensing elements comprises a graphene layer disposed on an underlying substrate. At least one of the sensing elements comprises a graphene layer functionalized with a plurality of anti-troponin antibodies and at least another one of the sensing elements comprises a graphene layer functionalized with a plurality of antibodies exhibiting a specific binding to a protein other than troponin.

In some embodiments, one of the sensing elements comprises a graphene layer functionalized with anti-troponin antibodies and another one of the sensing elements comprises a graphene layer functionalized with anti-CRP antibodies.

In some embodiments, one of the sensing elements comprises a graphene layer functionalized with anti-troponin antibodies and another one of the sensing elements comprises a graphene layer functionalized with anti-BNP antibodies.

In some embodiments, one of the sensing elements comprises a graphene layer functionalized with anti-troponin antibodies and another one of the sensing elements comprises a graphene layer functionalized with anti-CK-MB antibodies.

In some embodiments, one of the sensing elements comprises a graphene layer functionalized with anti-troponin antibodies and another one of the sensing elements comprises a graphene layer functionalized with anti-myoglobin antibodies.

In some embodiments, one of the sensing elements comprises a graphene layer functionalized with anti-troponin antibodies and another one of the sensing elements comprises a graphene layer functionalized with anti-MP antibodies.

Further understanding of various aspects of the invention can be obtained with reference to the following detailed description and associated drawings, which are described briefly below.

### BRIEF DESCRIPTION OF THE DRAWINGS

FIG. 1 schematically depicts a sensor according to an embodiment of the present invention for detecting troponin in a sample, which includes a graphene layer functionalized with anti-troponin antibodies,

FIG. 2 is a partial schematic top view of the sensor of FIG. 1 depicting a plurality of electrically conductive pads to measure an electrical property of the functionalized graphene layer,

FIG. 3A depicts a circuit diagram of an example of a voltage-measuring device that can be employed for measuring a voltage induced across an antibody-functionalized graphene layer in response to application of a current thereto,

FIG. 3B schematically depicts an analyzer in communication with the voltage-measuring device shown in FIG. 3A for receiving the voltage measured by the voltage-measuring device as well as the current applied to the antibody-functionalized graphene layer,

FIG. 3C depicts an example of implementation of the analyzer shown in FIG. 3A,

FIG. 4 schematically depicts another embodiment of a sensor according to the present teachings, which comprises a sample-testing sensing element and a calibration sensing element,

FIG. 5 schematically depicts another embodiment of a sensor according to the present teachings, which comprises one group of sensing elements configured to detect one isoform of troponin and another group of sensing elements configured to detect a different isoform of troponin,

FIG. 6A schematically depicts the sensor of FIG. 5 coupled to a fluidic device for distributing a sample among the sensing elements of the sensor,

FIG. 6B is a top schematic view of the fluidic device depicted in FIG. 6A, which includes a central fluidic channel for receiving a sample and a plurality of channels branching from the central channel for delivering portions of the sample to a plurality of sensing elements,

FIG. 7 schematically depicts an embodiment of a sensor according to the present teachings, and

FIG. 8 depicts signals generated by a plurality of exemplary sensors according to the present teachings, which were functionalized with either no antibody, mouse isotype control antibody (non-specific), and-Troponin 1 monoclonal and body or anti-Troponin 1 polyclonal antibody (all at 20 μg/ml) in response to exposure to 10 μg/ml Troponin 1 (A bar) or troponin-depleted serum (B bar).

### DETAILED DESCRIPTION

The present teachings are generally directed to a graphene-based sensor that can be functionalized with a plurality of antibodies for detecting a number of different biomarkers. As discussed in more detail below, a sample suspected of containing a biomarker of interest can be brought into contact with the antibody-functionalized graphene layer. If the biomarker of interest in present in the sample under investigation, the interaction of the biomarker with the antibody coupled to the graphene layer can cause a change in at least one electrical property of the underlying graphene layer, e.g., its DC electrical resistance. Such a change in the electrical property of the graphene layer can be measured and be correlated with the presence of the biomarker of interest in the sample. In some embodiments, a sensor according to the present teachings allows not only detecting the presence of a biomarker of interest in a sample under investigation, but also quantifying the amount of the biomarker in the sample, as discussed in more detail below.

The term “biomarker” as used herein refers to a molecular species with a biological function.

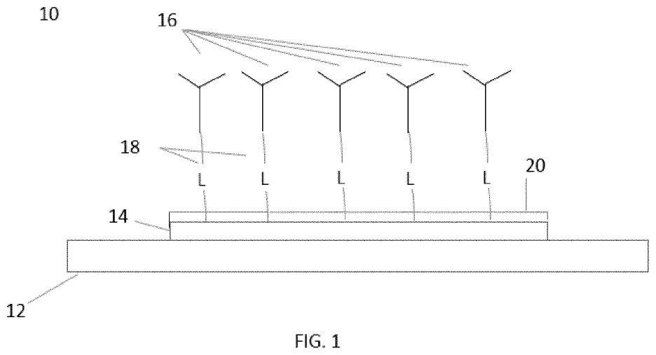

FIG. 1 schematically depicts a sensor 10 according to an embodiment of the present teachings for detecting a biomarker in a sample, e.g., a patient's blood. The sensor 10 comprises a graphene layer 12, which is disposed on an underlying substrate 14. The underlying substrate can be formed of a variety of different materials, such as, silicon, polymeric materials, such as polyurethane, polyethylene terephthalate, or glass, among others. In some embodiments, the graphene layer is disposed over an underlying silicon oxide (SiO₂) layer, which can in turn be formed as a thin layer in a silicon substrate (e.g., a layer having a thickness in a range of a 200 nm to about 10 microns).

In some embodiments, graphene can be deposited on an underlying silicon substrate by using a variety of techniques known in the art. By way of example, chemical vapor deposition (CND) can be employed to deposit graphene on an underlying copper substrate. The graphene-coated copper substrate can then be disposed on a silicon oxide layer of a silicon wafer, and the copper can be removed via chemical etching. In some embodiments, the graphene layer is deposited on the underlying substrate as an atomic monolayer, while in other embodiments the graphene layer includes multiple atomic layers.

In this embodiment, the graphene layer is functionalized with a plurality of antibodies 16 that exhibit specific binding to a biomarker of interest. By way of example, in this embodiment, the graphene layer is functionalized with a plurality of antibodies that exhibit specific binding to any of troponin, e.g., a particular isoform of troponin, C-reactive protein, B-type natriuretic peptide, or myeloperoxidase. In some embodiments, the anti-biomarker antibodies are monoclonal antibodies that exhibit specific binding to a particular isoform of the biomarker, e.g., a specific isoform of troponin. In other embodiments, the anti-biomarker antibodies can be polyclonal antibodies that exhibit binding to multiple isoforms of the biomarker. By way of example, in some embodiments, the graphene layer can be functionalized with cardiac troponin T (cTnT) and/or cardiac troponin I (cTnI).

As shown schematically in FIG. 1, a variety of linker molecules 18 can be employed for coupling the antibodies to the underlying graphene layer. By way of example, in some embodiments, 1-pyrenebutonic acid succinimidyl ester is employed as a linker to facilitate the coupling of the antibodies, e.g., anti-troponin antibodies, to the underlying graphene layer.

In this embodiment, the plurality of anti-troponin antibodies can cover a fraction of, or the entire, surface of the graphene layer. In various embodiments, the fraction can be at least about 60%, at least about 70%, at least about 80%, or 100% of the surface of the graphene layer. The remainder of the surface of the graphene layer (i.e., the surface areas not functionalized with the anti-biomarker antibodies) can be passivated via a passivation layer 20. By way of example, the passivation. layer can be formed by using Tween 20, BLOTTO, BSA (Bovine Serum Albumin), amino-PEG5-alcohol, and/or gelatin. The passivation layer can inhibit, and preferably prevent, the interaction of a sample of interest introduced onto the graphene layer with areas of the graphene layer that are not functionalized with the anti-biomarker antibodies. This can in turn lower the noise in the electrical signals that will be generated as a result of the interaction of the analyte of interest with the antibody molecules.

By way of example, in some embodiments, a graphene layer formed on an underlying substrate (e.g., plastic, a semiconductor, such as silicon, or a metal substrate, such as a copper film) can be incubated with the linker molecule (e.g., a 5 nM solution of 1-pyrenebutonic acid succinimidyl ester) for a few hours (e.g., 2 hours) at room temperature.

The linker modified graphene layer can then be incubated with the antibody of interest in a buffer solution (e.g., NaCO₃—NaHCO₃ buffer solution (pH 9)) at a selected temperature and for a selected duration (e.g., 7-10 hours at 4 C), followed by rinsing with deionized (DI) water and phosphate buffered solution (PBS). In order to quench the unreacted succinimidyl ester groups, the modified graphene layer can be incubated with ethanolamine (e.g., 0.1 M solution at a pH of 9 for 1 hour).

Subsequently, the non-functionalized graphene areas can be passivated via a passivation layer, such as the passivation layer 20 schematically depicted in FIG. 1. By way of example, the passivation of the non-functionalized portions of the graphene layer can be achieved, e.g., via incubation with 0.1% Tween 20.

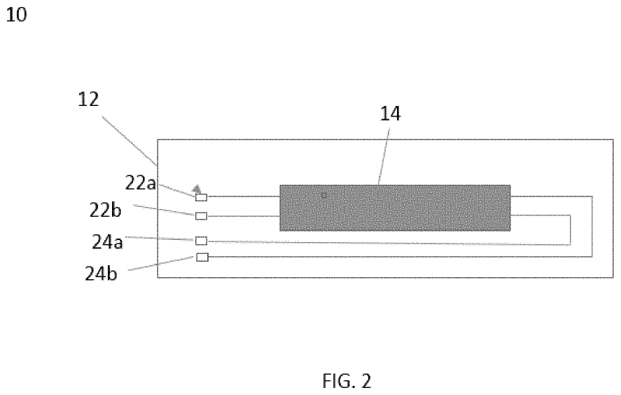

With reference to FIG. 2, the sensor 10 further includes electrically conductive pads 22a, 22b, 24a and 24b, that allow four point measurement of modulation of an electrical property of the functionalized graphene layer in response to interaction of the anti-biomarker antibodies with the troponin coupled to the graphene layer. In particular, in this embodiment, the conductive pads 22a /22b are electrically coupled to one end of the functionalized graphene layer and the conductive pads 24a /24b are electrically coupled to the opposed end of the functionalized graphene layer to allow measuring a change in an electrical property of the underlying graphene layer caused by the interaction of a biomarker of interest in a sample under study with the anti-biomarker antibodies, e.g., anti-troponin antibodies, that are coupled to the graphene layer. By way of example, in this embodiment, a change in the DC resistance of the underlying graphene layer can be monitored to determine the presence of the biomarker, e.g., troponin, in a sample under study. In other embodiments, a change in electrical impedance of the graphene layer characterized by a combination of DC resistance and capacitance of the graphene/antibody system can be monitored to determine whether a biomarker of interest is present in a sample under study. The electrically conductive pads can be formed using a variety of metals, such as copper and copper alloys, among others.

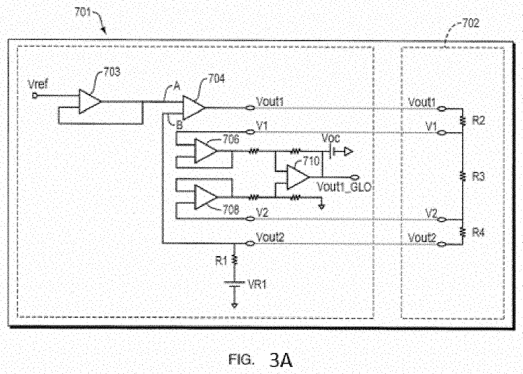

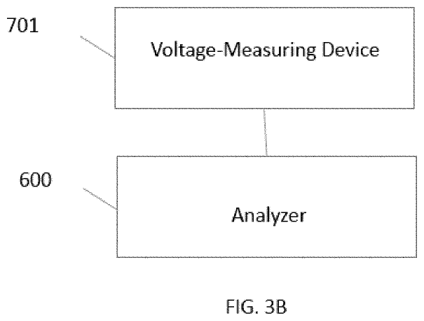

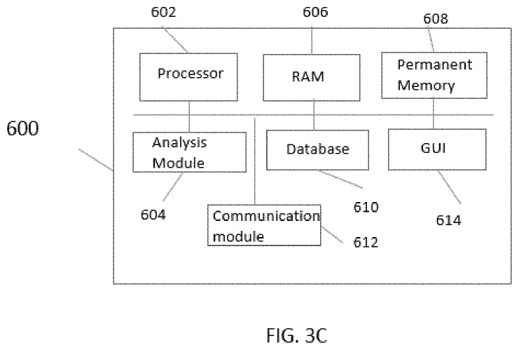

By way of example, FIG. 3A schematically depicts a voltage measuring circuitry 701 that can be employed in some embodiments of the present teachings. This figure shows a sensor 702 as an equivalent circuit corresponding to an antibody-functionalized graphene layer. A fixed voltage V (e.g., 1.2 V) is generated at the output of a buffer operational amplifier 703. This voltage is applied to one input (A) of a downstream operational amplifier 704 whose other input B is coupled to VR1 ground via a resistor R1. The output of the operational amplifier 704 (Vₒᵤₜ₁) is coupled to one end of the sensor 702 and the end of the resistor R1 that is not connected to VR1 ground is coupled to the other end of the sensor 702 (in this schematic diagram, resistor R2 denotes the resistance between two electrode pads at one end of the equivalent sensor 702, resistor R3 denotes the resistance of the graphene layer extending between two inner electrodes of the sensor, and resistor R4 denotes the resistance between two electrode pads at the other end of the sensor). As the operational amplifier maintains the voltage at the end of the resistor R1 that is not connected to VR1 ground at the fixed voltage applied to its input (A), e.g., 1.2 V, a constant current source is generated that provides a constant current flow through the sensor 702 and returns to ground via the resistor R1 and VR1.

The voltage generated across the antibody-functionalized graphene layer is measured via the two inner electrodes of the sensor. Specifically, one pair of the inner electrode pads is coupled to a buffer operational amplifier 706 and the other pair is coupled to the other buffer operational amplifier 708. The outputs of the buffer operational amplifiers are applied to the input ports of a differential amplifier 710 whose output port provides the voltage difference across the antibody-functionalized graphene layer. This voltage difference (Vₒᵤₜ₁−GLO) can then be used to measure the resistance exhibited by the antibody-functionalized graphene layer. The current forced through R3 is set by I=(Vref−VR1)/R1, where the value of VR1 is digitally controlled. For each value of current I, the corresponding voltage (Vout1_GLO) is measured and stored. The resistance of the antibody-functionalized graphene layer can be calculated as the derivative of the voltage, Vout1_GLO, with respect to current I, i.e., R=dV/dI.

As shown schematically in FIG. 3B, in some embodiments, an analyzer 600 can be in communication with the voltage measuring circuitry 701 to receive the applied current and the measured voltage value and use these values to calculate the resistance of the antibody-functionalized graphene layer. The analyzer can then employ the calculated resistance, e.g., a change in the resistance in response to exposure of the antibody-functionalized graphene layer to a sample under investigation, to determine, in accordance with the present teachings, whether the sample contains a biomarker of interest, such as those listed herein.

By way of example, as shown schematically in FIG. 3C, in this embodiment, the analyzer 600 can include a processor 602, an analysis module 604, a random access memory (RAM) 606, a permanent memory 608, a database 610, a communication module 612, and a graphical user interface (GUI) 614. The analyzer 600 can employ the communication module 612 to communicate with the voltage measuring circuitry 701 to receive the values of the applied current and the measured voltage. The communication module 612 can be a wired or a wireless communication module. The analyzer 600 further includes a graphical user interface (GUI) 614 that allows a user to interact with the analyzer 600.

The analysis module 604 can employ the values of a current applied to the antibody-functionalized graphene layer as well as the voltage induced across the graphene layer to calculate a change in the resistance of the antibody-functionalized graphene layer in response to exposure thereof to a sample under investigation. The instructions for such calculation can be stored in the permanent memory 608 and can be transferred at runtime to RAM 606 via processor 602 for use by the analysis module 604. In some embodiments, the database 610 can store calibration data that can be employed for determining whether a biomarker of interest is present in a sample under study. By way of example, the database 610 can store calibration data indicative of a temporal change in the electrical resistance of an antibody-functionalized graphene layer in response to exposure to a particular biomarker. A comparison of a measured temporal variation of a similar antibody-functionalized graphene layer exposed to a sample suspected of containing the biomarker with the calibrated response can be used to determine whether the biomarker is present in the sample. The GUI 614 can allow a user to interact with the analyzer 600.

In some embodiments, the analyzer 604 can include an ac (alternating current) source of current, which can apply an AC current having a known amplitude and frequency to the graphene layer. The analyzer 604 can further include an AC voltmeter circuitry for measuring the ac voltage induced across the graphene layer in response to the application of the ac current to the layer. By measuring the amplitude and/or phase shift of the induced ac voltage, the electrical impedance of the graphene layer can be determined in a manner known in the art.

Further details regarding a suitable analyzer that can be employed in the practice of some embodiments of the present teachings can be found, e.g., in U.S. Pat. No. 9664,674 titled “Device and Method for Chemical Analysis,” which is herein incorporated by reference in its entirety.

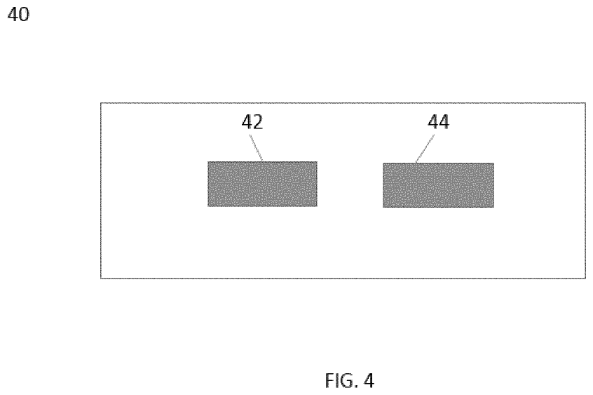

In some embodiments, a sensor according to the present teachings can allow quantifying the amount of a biomarker, if any, in a sample under study, e.g., a person's blood sample. By way of example, FIG. 4 schematically depicts such a sensor 40 that includes a test sensing element 42 and three calibration sensing elements 44a, 44b, and 44c. Each sensing element includes a graphene layer functionalized with antibodies exhibiting a specific binding to a biomarker of interest. e.g., anti-troponin antibodies, and has a structure similar to that discussed above in connection with the sensor 10.

In use, prior to testing a sample for presence of a biomarker, e.g., troponin., calibration samples having different concentrations of the biomarker, e.g., troponin, can be applied to the calibration sensing elements 44a, 44b, and 44c. Each calibration sample includes a different known amount of the biomarker, e.g., a known amount of an isoform of troponin such as cTNT or cTNI. An electrical signal generated by the calibration sensor in response to contact with the calibration sample can be measured and used for quantifying the amount of troponin in a sample under study. More specifically, each calibration sensing element 44a, 44b, and 44c can be used to obtain an electrical response of the functionalized graphene layer to troponin in one of the calibration samples. The responses of the three calibration sensing elements can be used to generate a calibration curve. While in this embodiment, three calibration sensing elements are employed, in other embodiments, the number of the calibration sensing elements can be more or less than three. A sample under study can be applied to the test sensing element 42, and a signal generated by the test sensing element can be compared with the calibration curve generated, for example, by employing at least three calibration data points, to quantify the amount of the biomarker, if any, in the sample under investigation.

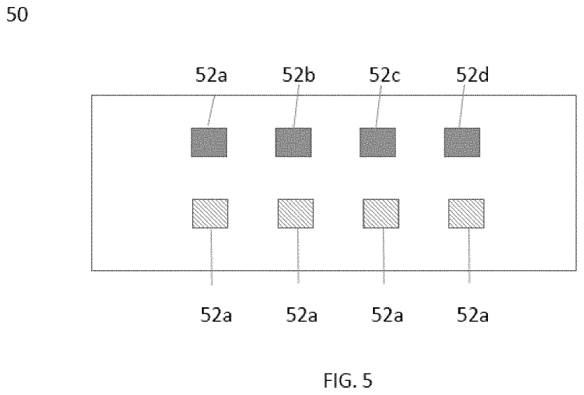

In some embodiments, a sensor according to the present teachings can include an array of sensing elements that allow parallel measurements of different isoforms of a biomarker, e.g., different isoforms of troponin. By way of example, FIG. 5 schematically depicts such a sensor 50 having a plurality of sensing elements 52a, 52b, 52c, and 52d (herein collectively referred to as sensing elements 52) as well as sensing elements 54a, 54b, 54c, and 54d (herein collectively referred to as sensing elements 54). Each of the sensing elements 52 and 54 includes a graphene layer functionalized with an isoform of a biomarker of interest, e.g., different isoforms of troponin, and has a structure similar to that discussed above in connection with sensor 10. By, way of example, in this embodiment, each of the sensing elements 52 is functionalized with one isoform of troponin and each of the sensing elements 54 is functionalized with a different isoform of troponin. By, way of example, each of the sensing elements 52 can be functionalized with cTNT while each of the sensing elements 54 can be functionalized with cTNI.

In some embodiments, each of the sensing element 52 and 54 can have an associated calibration sensor. Which can be employed to calibrate the corresponding sensing element in a manner discussed above.

A sample under study can be concurrently distributed among the sensing elements 52 and 54. The sensing elements 52 can determine whether the sample contains cTNT, and optionally quantify the amount of cTNT in the sample, and the sensing elements 54 can determine whether the sample contains cTNI, and optionally quantify the amount of cTNI in the sample.

An analyzer (not shown) can be used to measure the change in the resistances of the sensing elements 52 and 54 in response to contact of those sensing elements with a sample under study. In some embodiments, the analyzer can employ a multiplexing circuitry to measure sequentially the resistance of each of the sensing elements.

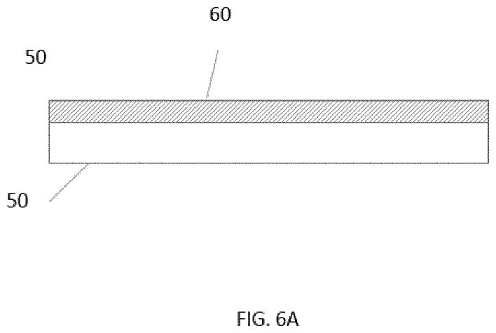

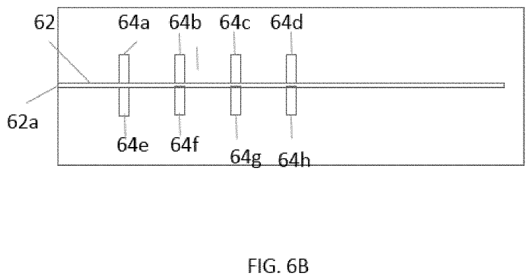

In some embodiments of the sensor 50, a fluidic delivery device can be employed to deliver a sample under study to the sensing elements 52 and 54. By way of example, FIGS. 6A and 6B schematically depict such a fluidic delivery device 60 that is fluidically coupled to the sensing elements 52 and 54. The fluid delivery device 60 includes a central capillary channel 62 having an input port 62a for receiving a sample and a plurality of peripheral capillary channels 64a, 64b, 64c 64d, 64e, 64f, 64g, and 64h for delivering the sample to the sensing elements 52 and 54.

In some embodiments, one or more of the sensing elements of the sensor 50 can be functionalized with antibodies exhibiting specific binding to C reactive protein (CRP) (herein referred to as anti-CRP antibodies), one or more of the other sensing elements can be functionalized with anti-bodies exhibiting specific binding to B-type natriuretic peptide (BNP) (herein referred to as anti-BNP antibodies), and one or more of the sensing elements can be functionalized with anti-troponin, e.g., anti-cTNT, antibodies. A sample under study, e.g., a blood or urine sample, can be introduced onto these sensing elements. One or more electrical signals generated by the sensing elements, e.g., signals generated due to a change in the resistance of the underlying graphene layer, in response to the interaction of the sample with the functionalized graphene layer can be detected and analyzed to identify the presence of the above proteins, i.e CRP, BNP and troponin, in the sample under study.

As discussed above, in some embodiments, each sensing element can have an associated calibration sensing element that allows calibrating the sensing element for quantifying the concentration of a protein in a sample under study. In this manner, a sensor having a plurality of sensing elements functionalized with a variety of different biomarkers, such as the above biomarkers (CRP, B-type natriuretic peptide and CTNT), can be used to not only identify the presence of that biomarker in a sample but also quantify its concentration.

A panel of a plurality of biomarkers can be used as a diagnostic tool for diagnosing one or more disease conditions. Further, in some cases, a panel of biomarkers can be used as a predictive tool. For example, it has been reported that a combination of cTNT, BNP and CRP can be used as a prognostic tool to predict long-term mortality in haemodialysis (HD) patients.

In another embodiment, one or more of the sensing elements of the sensor 50 can be functionalized with anti-bodies exhibiting specific binding to troponin I, e.g., cardiac troponin I, one or more of the other sensing elements can be functionalized with antibodies exhibiting specific binding to myoglobin, e.g., monoclonal or polyclonal anti-myoglobin antibodies available from Invitrogen, and one or more of the sensing elements can be functionalized with anti-bodies exhibiting specific binding to creatine kinase MB (CK-MB).

A sample, e.g., anticoagulated whole blood or a plasma specimen, can be brought into contact with the sensing elements, e.g., concurrently or sequentially, to determine the level of each of the above proteins, i.e., cTNI, myoglobin and CK-MB, in the specimen. Similar to the previous embodiment, a plurality of calibration sensors functionalized in a manner similar to the testing sensors can be used to quantify the amount of any of these proteins in the sample. By way of example, such a multi-panel test can be used as an aid in the diagnosis of myocardial infarction.

In some embodiments, one or more of the sensing elements of the above sensor 50 can be functionalized with anti-myeloproxidase (anti-MP) antibodies to detect, for example, the presence of myeloperoxidase in a biological sample, e.g., blood. In sonic embodiments, such a sensing element can be used in conjunction with other sensing elements, e.g., a sensing element functionalized with anti-CRP antibodies, as an aid in assessing risk of adverse cardiac events. In some embodiments, a sensor according to the present teachings can detect any of the above analytes, e.g., various isoforms of troponin, at a concentration as low as 5 μg/ml, or as low as 1 μg/ml, or as low as 1 picogram/ml.

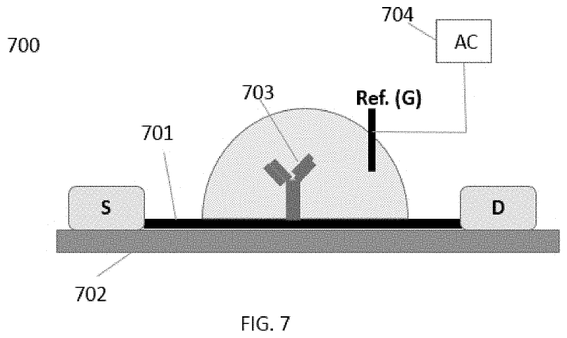

FIG. 7 schematically depicts another embodiment of a sensor 700 according to the present teachings, which includes a graphene layer 701 that is disposed on an underlying substrate 702, e.g., a semiconductor substrate, and is functionalized with an antibody of interest 703. A source electrode (S) and a drain electrode (D) are electrically coupled to the graphene layer to allow measuring a change in one or more electrical parameters of the functionalized graplene layer in response to interaction of the functionalized graphene layer with a sample. The sensor 700 further includes a reference electrode (G) that is disposed in proximity of the graphene layer.

In use, in some embodiments, a change in the electrical resistance of the functionalized graphene layer can be measured in response to the interaction of the functionalized graphene layer with a sample to identify and optionally quantify an analyte of interest, e.g., a biomarker, in the sample. For example, when the sample includes an analyte that specifically binds to the antibody 703, the interaction of the antibody with the analyte can modulate the electrical resistance of the graphene layer. A measurement of such a modulation of the electrical resistance of the graphene layer can be employed to identify that analyte in a sample.

It has been discovered that the application of an AC (alternating current) voltage via an AC voltage source 704 to the graphene layer can facilitate the detection of one or more electrical properties of the functionalized graphene, e.g., a change in its resistance in response to the interaction of the antibody with an analyte exhibiting specific binding to the antibody. In particular, it has been discovered that the application of an AC voltage having a frequency in a range of about 1 kHz to about 1 MHz, e.g., in a range of about 10 kHz to about 500 kHz or in a range of about 20 kHz to about 100 kHz or in a range of about 30 kHz to about 60 kHz or in a range of about 100 kHz to about 200 kHz or in a range of about 200 kHz to about 300 kHz or in a range of about 400 kHz to about 500 kHz, can be especially advantageous in this regard. By way of example, the amplitude of the ac voltage applied to the reference electrode can be in a range of about 1 millivolt to about 3 volts, e.g., 0.5 volts to 1 volt. Further, in some cases, the voltage applied to the reference electrode can have an ac component and a dr offset, where the DC offset can be in a range of about −40 volts to about +40 volts, e.g., −1 volt to about +1 volt.

Without being limited to any particular theory, in some embodiments, it is expected that the application of a such a voltage to the reference electrode can minimize, and preferably eliminate, an effective capacitance associated with a sample, e.g., a liquid sample, with which the functionalized graphene layer is brought into contact as the sample is being tested, thereby facilitating the detection of a change in the resistance of the underlying graphene layer in response to the interaction of the antibodies 703 with a respective antigen. In some cases, the effective capacitance of the sample can be due to ions present in the sample.

The following example are provided for further elucidation of various aspects of the invention. The examples are provided only for illustrative purposes and not for necessarily indicating optimal ways of practicing the invention and/or optimal results that can be obtained.

Antibodies that exhibit binding to troponin. C-reactive protein, myeloperoxidase and B-type natriuretic peptide (BNP) suitable for use in the practice of the invention are commercially available. For example, antibodies that exhibit specific binding to myeloperoxidase can be obtained from abcam (ab9535) and B-type natriuretic peptide (BNP) can also be obtained from abcam (ab19645). Further, anti-cardiac troponin I antibody and anti-cardiac troponin T antibody can be obtained from Abcam (ab47003, and ab8295, respectively). In addition, anti-C reactive protein antibodies can also be acquired from Abeam (ab 31156).

### EXAMPLE 1

A plurality of sensors according to the embodiment depicted in FIG. 7 were functionalized with either no antibody, isotype control antibody, anti-Troponin I monoclonal antibody or anti-Troponin 1 polyclonal antibody (all at 20 μg/ml). 10 μg/ml Troponin 1 (A bar) or troponin-depleted serum (B bar) were applied to the antibody-bound sensors. Troponin 1 and anti-troponin antibodies (both monoclonal and polyclonal) were provided by Lumos Diagnostics. To facilitate the detection of a change in the resistance of the graphene layer, a voltage having an AC component at a frequency of 100 kHz, and an amplitude of 5 millivolts, and a DC offset of 0.02 volts, was applied to the reference electrode of the sensor.

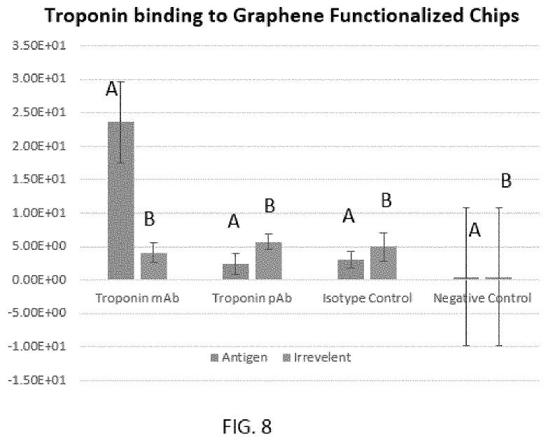

As shown in FIG. 8, the sensors functionalized with the monoclonal anti-troponin antibody produced signals indicative of the interaction of troponin with the antibody bound to the sensors' graphene layer. But the sensors functionalized with the polyclonal antibody did not show any binding above that exhibited by the sensors functionalized with isotype control antibody. As the sensor functionalized with the polyclonal antibody was expected to exhibit a stronger signal, this result needs to be further investigated. For example, it is possible that the polyclonal antibody that was employed in this experiment was not the correct antibody for the isoform of troponin that was tested or the polyclonal antibody had degraded and was no longer functional. The sensors can be further optimized for improving the detection limit for detecting troponin.

Those having ordinary skill in the art will appreciate that various changes can be made to the above embodiments without departing from the scope of the invention.

1. A sensor for detecting a biomarker in a sample, comprising:
graphene layer,
a plurality of antibodies coupled to said graphene layer, said antibodies exhibiting specific binding to said biomarker,
a reference electrode disposed in vicinity of said graphene layer,
an AC voltage source for applying an AC voltage to said reference electrode so as to generate a time-varying electric field at said graphene surface, and
a plurality of electrical conductors electrically coupled to said functionalized graphene layer for measuring an electrical property of said functionalized graphene layer.

2. The sensor of claim 1, wherein said ac voltage source is configured to apply an ac voltage with a frequency in a range of about 1 kHz to about 1 MHz.

3. The sensor of claim 2, wherein said applied ac voltage has an amplitude in a range of about 1 millivolt to about 3 volts.

4. The sensor of claim 1, wherein said biomarker comprises any of troponin C-reactive protein, B-type natriuretic peptide, myeloperoxidase, and creatine kinase MB.

5. A system for detecting a panel of biomarkers in a biological sample, comprising:
a plurality of sensors each configured to detect one of a plurality of biomarkers, each of said sensors comprising:
a graphene layer disposed on an underlying substrate,
a plurality of antibodies coupled to said graphene layer, wherein said antibodies exhibit specific binding to one of said plurality of biomarkers,
a plurality of electrical conductors electrically coupled to said antibody-functionalized graphene layer to measure at least one electrical property of the antibody-functionalized graphene layer.

6. The system of claim 5, wherein at least one of said sensors is configured for detecting troponin in said biological sample.

7. The system of claim 5, wherein at least one of said sensors is configured for detecting reactive protein in said biological sample.

8. The system of claim 5, wherein at least one of said sensors is configured for detecting B-type natriuretic peptide in said biological sample.

9. The system of claim 5, wherein at least one of said sensors is configured for detecting myeloperoxidase in said biological sample.

10. The system of claim 5, wherein at least one of said sensors is configured for detecting creatine kinase MB in said biological sample.

11. A sensor for detecting a biomarker in a biological sample, comprising:
a test sensing unit configured for receiving said biological sample and detecting said biomarker in said biological sample if a concentration of the biomarker in the sample is above a threshold,
a calibration sensing unit configured for receiving a calibration sample for providing calibration data for use in quantifying the biomarker detected by said test sensing unit,
wherein each of said test sensing unit and the calibration sensing unit comprises:
a graphene layer disposed on an underlying substrate,
a plurality of antibodies coupled to said graphene layer, wherein said antibodies exhibiting specific binding to said biomarker, and.
a plurality of electrical conductors electrically coupled to said functionalized graphene layer for measuring an electrical property of said functionalized graphene layer.

12. The sensor of claim 11, wherein said biomarker comprises any of troponin, C-reactive protein, B-type natriuretic peptide, and myeloperoxidase.

13. The sensor of claim 12, wherein said biological sample comprises any of blood, urine, saliva, spinal fluid, vaginal secretions, fecal slurries, and pleural lavage fluid.

14. The sensor of claim 11, further comprising an analyzer for receiving data from said test sensing unit and said calibration sensing unit and operating on said data to quantify amount of said biomarker in said biological sample.

15. The sensor of claim 11, wherein each of said test sensing unit and said calibration testing unit comprises a reference electrode disposed in proximity of said graphene layer.

16. The sensor of claim 15, further comprising an ac voltage source for applying an ac voltage to said reference electrode.

17. The sensor of claim 16, wherein said ac voltage has a frequency in a range of about 1 kHz to about 1 MHz.

18. The sensor of claim 17, wherein said ac voltage has an amplitude in a range of about 1 millivolt to about 3 volts.

19. A sensor for detecting troponin in a sample, comprising:
a graphene
a plurality of anti-troponin antibodies coupled to said graphene layer to generate a functionalized graphene layer, and
a plurality of electrical conductors electrically coupled to said functionalized graphene layer for measuring an electrical property of said functionalized graphene layer.

20. The sensor of claim 19, wherein said anti-troponin antibodies comprise antibodies exhibiting specific binding to cardiac troponin I protein.

21. The sensor of claim 19, wherein said anti-troponin antibodies comprise antibodies exhibiting specific binding to cardiac troponin T protein.

22. The sensor of claim 19, wherein said graphene layer is disposed on an underlying substrate.

23. The sensor of claim 22, wherein said underlying substrate comprises a semiconductor.

24. The sensor of claim 23, wherein said semiconductor comprises silicon.
